(19) United States
(12) Patent Application Publication
(10) Pub. No.: US 2007/0156173 A1
(43) Pub. Date: Jul. 5, 2007

(54) Earwax extracting apparatus and method comprising same

(76) Inventors: Don Alan Carnahan, Warrenton, OR (US); Tami Maylene Carnahan, Warrenton, OR (US)

Correspondence Address: David O. Simmons;Galasso & Associates LP, P.O. Box 26503, Austin, TX, 78755-0503, US

(21) Appl. No.: 11/321,173

(22) Filed: Dec. 29, 2005

(51) Int. Cl. A61B 17/50; A61B 1/267

(52) U.S. Cl. 606/211; 600/200

(57) ABSTRACT

An earwax extracting apparatus comprises a housing, a tapered body and spaced apart extractor arms. The housing includes a first opening in a first face thereof, a second opening in a second face thereof and an interior space extending between the first opening and the second opening. The first face is generally opposite the second face. The tapered body is attached to the housing over the first opening. The tapered body has a viewing passage extending therethrough. A generally straight line of sight extends through the viewing passage, the first opening of the housing and the second opening of the housing. The spaced apart extractor arms are movably attached to the housing. Each one of said extractor arms is movable between a respective retracted position and a respective extended position with respect to a tip portion of the tapered body. The tapered body extends longitudinally between said spaced apart extractor arms.

### FIELD OF THE DISCLOSURE

The disclosures made herein relate generally to medical apparatuses and, more particularly, to medical apparatuses configured for extracting earwax from an ear.

### BACKGROUND

People perform certain acts of hygiene in order for their body to remain in a healthy physical state. Examples of these acts include cleaning ears, brushing teeth, showering, bathing, applying moisturizers, brushing hair, shaving, washing hair and trimming finger/toe nails. Some of these acts are performed daily while others are performed less than daily. Factors such as personal preferences, aspects relating to a specific act, activities performed by a person and the like will dictate the specific frequency in which various acts of hygiene are performed.

Earwax is typically removed through the use of a cotton swab. Many doctors now recommend, however, that a person use only his or her finger to perform the task. The reason for this recommendation is that a cotton swab may be pushed too far into the ear and damage the eardrum. This potential for damaging the eardrum is especially true when a parent cleans the ear of their child, as they cannot feel how far the cotton swab is going into the ear canal.

Often times, a medical professional such as a physician must remove earwax because it has become impacted inside the ear canal. The tool traditionally used for viewing inside of the ear canal is an otoscope. The otoscope includes a magnifying lens and light, which provides for a detailed inspection within the ear canal. While this tool is effective for viewing inside of the ear canal, conventional otoscopes do not include any integral means for removing earwax. Thus, after using the otoscope for determining that there is earwax that must be removed, the medical professional removes the otoscope and does a ‘blind sweep’ of the ear using a separate apparatus (e.g., a cotton swap) to retrieve earwax.

Currently, medical professionals simply do not have the visibility that they would otherwise desire when performing such a delicate procedure as removing earwax from a patient's ear canal. Therefore, an apparatus that is configured for removing earwax from an ear canal and that overcomes shortcomings associated with conventional implements and/or methods suitable for removing earwax from an ear canal would be useful and advantageous.

### SUMMARY OF THE DISCLOSURE

Embodiments of the present invention are suitably configured for enabling earwax and other substances and/or foreign objects to be conveniently and safely extracted from a patient's ear canal and/or other body cavity or passage. More specifically, embodiments of the present invention are particularly well-suited for removing earwax from a patient's ear canal. An earwax extracting apparatus in accordance with the present invention has an invasive portion that is insertable into a patient's ear canal or other body cavity or passage. Specific to earwax extraction, the invasive portion is configured for enabling a user to view the ear canal and for extracting earwax while viewing such earwax. In doing so, embodiments of the present invention advantageously overcome one or more shortcomings associated with conventional approaches and tools used for removing earwax from a patient's ear canal.

In one embodiment of the present invention, an extraction apparatus comprises a housing, a tapered body and spaced apart extractor arms. The housing includes a first opening in a first face thereof, a second opening in a second face thereof and an interior space extending between the first opening and the second opening. The first face is generally opposite the second face. The tapered body is attached to the housing over the first opening. The tapered body has a viewing passage extending therethrough. A generally straight line of sight extends through the viewing passage; the first opening of the housing and the second opening of the housing. Each one of the spaced apart extractor arms is movably attached to the housing. Each one of the extractor arms is movable between a respective retracted position and a respective extended position with respect to a tip portion of the tapered body. The tapered body extends longitudinally between the spaced apart extractor arms.

In another embodiment of the present invention, an earwax extracting otoscope comprises a head assembly, a handle assembly and an optical fiber. The head assembly includes a head assembly housing, a magnifying lens attached to the head assembly housing, spaced apart extractor arms movably attached to the head assembly housing and a tapered body attached to the head assembly housing. A generally straight line of sight extends through the magnifying lens and a viewing passage of the tapered body. Each one of the extractor arms is movable between a respective retracted position and a respective extended position with respect to a tip portion of the tapered body. The tapered body extends longitudinally between the spaced apart extractor arms. The handle assembly includes a handle assembly housing attached to the head assembly housing and a light source mounted within the handle assembly housing. The optical fiber is attached between the light source and the head assembly housing for enabling a beam of light to be directed from the optical fiber through the viewing passage of the tapered body.

In another embodiment of the present invention, a method for extracting earwax from within an ear canal comprises a plurality of operations. An operation is performed for inserting an invasive portion of an earwax extracting apparatus into an ear canal of a patient. The invasive portion includes a respective tip portion of spaced apart extractor arms and a tip portion of a tapered body disposed between the extractor arms. An operation is performed for directing a beam of light through a viewing passage of the tapered body into the ear canal for enabling earwax therein to be viewed through the viewing passage of the tapered body. An operation is performed for causing the extractor arms to displace with respect to the tip portion of the tapered body for bringing the extractor arms into engagement with earwax in the ear canal and an operation is performed for extracting the invasive portion of the earwax extracting apparatus from within the ear canal.

Turning now to specific aspects of the present invention, in at least one embodiment, each one of the extractor arms is biased to the respective retracted position and the respective extractor arms is biased against an exterior face of the tapered body such that movement of the extractor arms toward the extended position causes each one of the extractor arms to follow a profile of the tapered body whereby tip portions of the extractor arms move closer together as each one of the extractor arms is moved from the respective retracted position toward the respective extended position.

In at least one embodiment of the present invention, the tip portions of the extractor arms remain within a field of view through the viewing channel of the tapered body when each one of the extractor arms is moved between the respective retracted position and the respective extended position.

In at least one embodiment of the present invention, wherein the magnifying lens is mounted within a tip portion of the tapered body.

In at least one embodiment of the present invention, the magnifying lens is mounted within an opening of the head assembly housing and the line of sight extending through the viewing passage of the tapered body and the opening in the head assembly housing.

In at least one embodiment of the present invention, the head assembly includes an extractor arm actuation member movably attached to the housing and movement of the extractor arm actuation member from an at-rest position toward a displaced position causes each one of the extractor arms to move from the respective retracted position toward the respective extended position.

In at least one embodiment of the present invention, causing the extractor arms to displace includes moving an extractor arm actuation member from an at-rest position:toward a displaced position.

In at least one embodiment of the present invention, moving the extractor arms includes maintaining respective tip portions of the extractor arms within a field of view through the viewing channel of the tapered body when each one of the extractor arms is moved between the respective retracted position and the respective extended position.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a perspective view of an embodiment of an earwax extracting otoscope in accordance with the present invention.

FIG. 2 is a rear view of the earwax extracting otoscope depicted in FIG. 1.

FIG. 3 is a cross-sectional view taken along the line 3-3 in FIG. 2.

FIG. 4 is an enlarged partial fragmentary view depicting an alternate embodiment of an invasive portion of an earwax extracting apparatus in accordance with the present invention.

### DETAILED DESCRIPTION OF THE DRAWING FIGURES

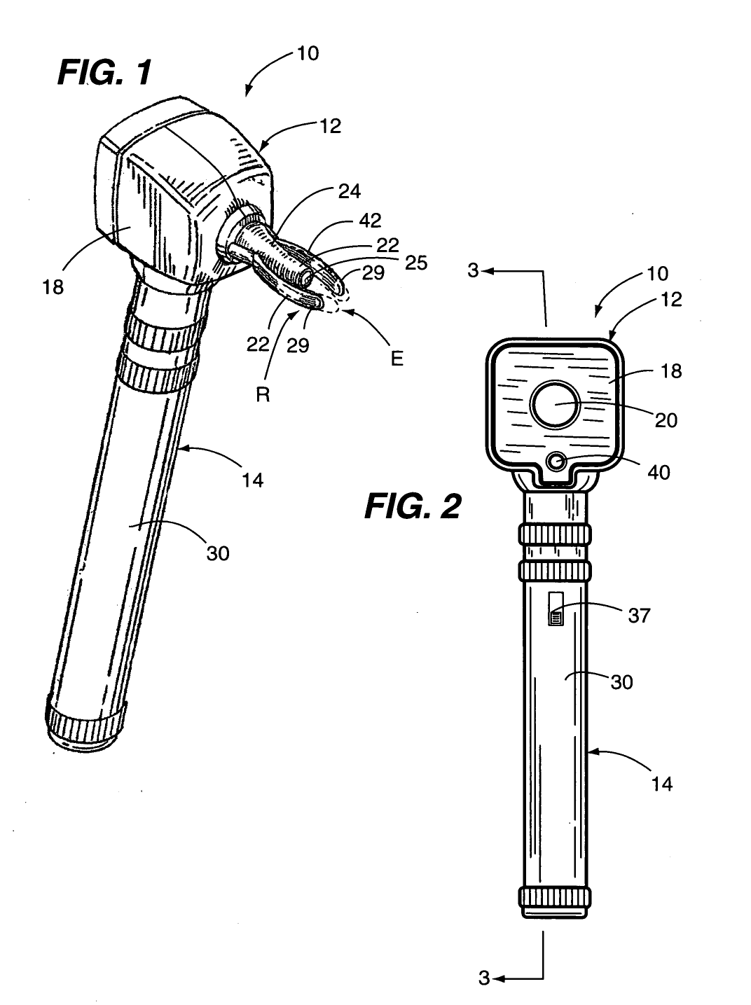

FIGS. 1-3 depict an embodiment of an earwax extracting otoscope in accordance with the present invention, which is referred to herein as the otoscope 10. The earwax extracting otoscope 10 includes an invasive portion that is insertable into a patient's ear canal. The invasive portion is configured for enabling a user to view the ear canal and for extracting at least a portion of earwax from within the canal while viewing such earwax. In doing so, embodiments of the present invention advantageously overcome one or more shortcomings associated with conventional approaches and tools used for removing earwax from a patient's ear canal. In view of the disclosures made herein, a skilled person will appreciate that an earwax extracting otoscope is a specific embodiment of an extraction device in accordance with the present invention. Such an extraction device may be used specifically as disclosed or modified within the scope of the disclosures made herein to be usefull in extraction applications besides earwax extraction (e.g., extracting items from a patient's nose).

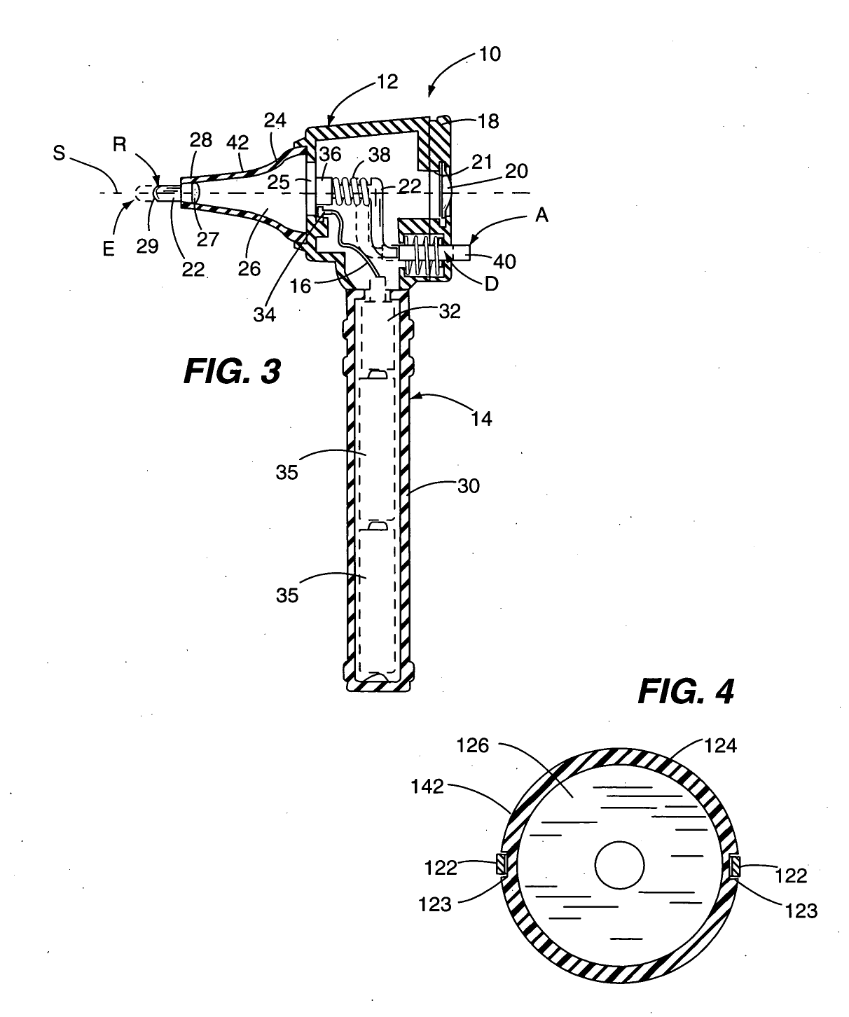

The otoscope 10 includes a head assembly 12, a handle assembly 14 and an optical fiber 16 (FIG. 3). It is disclosed herein that the head assembly 12 is an example of an earwax extracting apparatus in accordance with the present invention. The head assembly 12 includes a head assembly housing 18, a first lens 20 attached to the head assembly housing 18 within a first opening 21 of the head assembly housing 18, two spaced apart extractor arms 22 movably attached to the head assembly housing 18, a tapered body 24 attached to the head assembly housing 18 over a second opening 25 of the head assembly housing 18 and a second lens 27 attached within a viewing passage 26 of the tapered body 24. A generally straight line of sight S extends through the first lens 20, the viewing passing 26 (FIG. 3) of the tapered body 24 and the second lens 25. The two spaced apart extractor arms 22 is 180 degrees separated (i.e., on opposing sides of the tapered body 24).

Preferably, the first lens 20 or the second lens 25 is a magnifying lens. Optionally, both the first lens 20 and the second lens 25 are magnifying lenses. The tapered body 24 functions as a speculum that aids in opening the ear canal of a patient.

Each one of the extractor aims 22 is movable between a respective retracted position R and a respective extended position E with respect to a tip portion 28 of the tapered body 24. The tapered body 24 extends longitudinally between the two spaced apart extractor arms 22. A respective tip portion 29 of each one of the extractor arms 22 extends beyond the tip portion 28 of the tapered body 24 by a first distance when in the respective retracted position R and by a second distance greater than the first distance when in the respective extended position E. An invasive portion of an earwax extracting apparatus in accordance with the present invention is disclosed herein to include the tip portions 29 of the extractor arms 22 and the tip portion 28 of the tapered body 24.

The handle assembly 14 includes a handle assembly housing 30 and a light source 32 (FIG. 3). The handle assembly housing 30 is attached to the head assembly housing 18. The light source 32 is mounted within the handle assembly housing 30. The optical fiber 16 attached between the light source 32 and the head assembly housing 18 for enabling a beam of light to be directed from an end 34 of the optical fiber through the viewing passage 26 of the tapered body 24. A conventional LED light source is an example of the light source 32. A power source such as batteries 35 is provided within the handle assembly housing 18 for providing electrical power to the light source 32. A switch 37 is electrically connected between the power source and the light source 32 for enabling electrical power to be selectively provided from the power source to the light source 32. It is disclosed herein that the present invention is not limited to a particular electrical configuration for the light source and/or power source. Accordingly, in general, suitable proprietary and/or known electrical circuitry (e.g., wires, stamped leads and/or switches) provide for selective routing of electrical power from the power source of the otoscope 10 to the light source 32.

Each extractor arm 22 is slideably engages with a respective mounting structure 36 and is biased to the respective retracted position R by a respective resilient member 38. Each mounting structure 36 is mounted at a perimeter location of the head assembly housing 18 such that each, mounting structure 36 is not within the line of sight through the viewing passage 26 of the tapered body 24. A helical spring is an example of the resilient member 38.

The head assembly 12 includes an extractor arm actuation member 40 that is slideably attached to the head assembly housing 18 and is engaged with each extractor arm 22 (e.g., in abutted engagement). Movement of the extractor arm actuation member 40 from an at-rest position A toward a displaced position D applies a force on the extractor arms 22, which causes the extractor arms 22 to simultaneously move from the respective retracted position R toward the respective extended position E. While FIG. 3 shows the specific mounting arrangement and actuation member engagement arrangement for only one of the extractor arms 22 due to the cross sectioning location, the specific mounting arrangement and actuation member engagement arrangement is the same for both of the extractor arms 22.

It is disclosed herein that the present invention is not limited to the disclosed mounting and engagement arrangements. More specifically, an earwax extracting apparatus in accordance with the present invention includes a suitable configuration for enabling actuation of the extractor arms 22. The mounting arrangement and actuation member engagement arrangement depicted in FIG. 3 is one such suitable configuration.

Each one of the extractor arms 22 is biased against an exterior face 42 of the tapered body 24. Preferably, but not necessarily, the extractor arms 22 are themselves resilient and are mounted on the head assembly body 18 such that the extractor arms 22 exert a biasing force on the exterior face 42 of the tapered body 24 as each one of the extractor arms 22 moves between the respective retracted position R and respective extended position E. More specifically, movement of each one of the extractor arms 22 from the respective retracted position R toward the respective extended position E causes each one of the extractor arms 22 to follow a profile of the exterior face 42 during such movement from the respective retracted position R toward the respective extended position E. Accordingly, due in part to biasing against the exterior face 42 of the tapered body 24 and the tapered profile of the exterior face 42, the respective tip portions 29 of each one of the extractor arms 22 move closer together (i.e., a pinching action) as each one of the extractor arms 22 is moved from the respective retracted position R toward the respective extended position E.

Optionally, the head assembly 12 is configured such that the extractor arms are not biased against the exterior face 42 of the tapered body 24. Accordingly, in such an embodiment, the respective tip portions 29 of each one of the extractor arms 22 do not appreciably move closer together as each one of the extractor arms 22 is moved from the respective retracted position R toward the respective extended position E.

The tip portion 29 of each one of the extractor arms 22 remains within a field of view through the viewing channel 26 of the tapered body 24 (i.e., as seen looking through the head assembly housing) when each one of the extractor arms 22 is moved between the respective retracted position R and the respective extended position E. In doing so, a medical professional using the earwax extracting otoscope 10 is able to precisely and accurately control placement of the tip portions 29 of the extractor arms while the tip portions 29 are within a patient's ear canal.

Presented now are specific, but not limiting, details of the head assembly 12 of the earwax extracting otoscope 10. The tapered body 24 is preferably approximately 3.5 cm long and is tapered from a diameter of about 1.2 cm to about 3 mm at its tip portion 28. In their respective retracted positions R, each one of the extractor arms 22 extend beyond the tip portion 28 of the tapered body 24 by a maximum distance of approximately 1 cm. The tip portion 29 of the extractor arms 22 is generally flat having a thickness of about 1 to 2 mm and a width of about 2.5 to 3 mm, thus enabling ease of insertion into a patient's ear canal. The differential in distance between the retraced position R and the extended position E is approximately 0.9 cm.

In at least one embodiment of the present invention, each one of the extractor arms 22 is removably attached to the head assembly housing 18 to allow for cleaning between uses. Additionally, sets of extractor arms having different material configuration, physical configuration and/or other characteristics may be provided. For example, extractor arms 22 having differing hardness material, different shape tip portions, different level of resiliency, etc may be interchangeably attached to the head assembly housing 18.

FIG. 4 depicts an embodiment of a head assembly 112 in accordance with the present invention in which extractor arms 122 each reside in a respective groove 123 in a tapered body 124 when the extractor arms 122. Preferably, but not necessarily, an exterior face 142 of the tapered body 124 has a straight (i.e., non-curved taper). Preferably, but not necessarily, the extractor arms 122 lie approximately or fully flush with the exterior face 142 of the tapered body 124.

Turning now to a description of a method of use of the otoscope 10, an operation is performed for inserting the invasive portion of the earwax removing apparatus into an ear canal of a patient. As disclosed above, the invasive portion includes the tip portion 29 of the extractor arms 22 and the tip portion 28 of the tapered body 24. An operation is performed for directing a beam of light through the viewing passage 26 of the tapered body 24 into the ear canal for enabling earwax therein to be viewed through the viewing passage 26 of the tapered body 24. After identifying earwax that is to be extracted, an operation is performed for causing the extractor arms 22 to displace with respect to the tip portion 28 of the tapered body 24 for bringing the tip portion 29 of the extractor arms 22 into engagement with the earwax. Causing the extractor arms 22 to displace includes moving the extractor arm actuation member 40 from the at-rest position A toward the displaced position D thereby causing each one of the extractor arms 22 to move from their respective retracted position R toward their respective extended position E. Thereafter, an operation is performed for extracting the invasive portion of the earwax extracting apparatus from within the ear canal.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes maybe made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. An extraction apparatus, comprising:
a housing including a first opening in a first face thereof, a second opening in a second face thereof and an interior space extending between the first opening and the second opening, wherein the first face is generally opposite the second face;
a tapered body attached to the housing over the first opening, wherein the tapered body has a viewing passage extending therethrough and wherein a generally straight line of sight extends through the viewing passage, the first opening of the housing and the second opening of the housing; and
spaced apart extractor arms movably attached to the housing, wherein each one of said extractor arms is movable between a respective retracted position and a respective extended position with respect to a tip portion of the tapered body and wherein the tapered body extends longitudinally between said spaced apart extractor arms.

2. The extraction apparatus of claim 1 wherein:
each one of said extractor arms is biased to the respective retracted position; and
each one of said extractor arms is biased against an exterior face of the tapered body such that movement of each one of said extractor arms toward the respective extended position causes each one of said extractor arms to follow a profile of the tapered body whereby tip portions of each one of said extractor arms move closer together as each one of said extractor arms is moved from the respective retracted position toward the respective extended position.

3. The extraction apparatus of claim 2 wherein said tip portions of each one of said extractor arms remain within a field of view through the viewing channel of the tapered body when each one of said extractor arms is moved between the respective retracted position and the respective extended position.

4. The extraction apparatus of claim 1 wherein tip portions of said extractor arms remain within a field of view through the viewing channel of the tapered body when each one of said extractor arms is moved between the respective retracted position and the respective extended position.

5. The extraction apparatus of claim 1, further comprising:
a magnifying lens mounted within a tip portion of the tapered body.

6. The extraction apparatus of claim 1, further comprising:
a magnifying lens mounted within the second opening of the housing.

7. The extraction apparatus of claim 1, further comprising:
an extractor arm actuation member movably attached to the housing, wherein movement of the extractor arm actuation member from an at-rest position toward a displaced position causes each one of said extractor arms to move from the respective retracted position toward the respective extended position.

8. The extraction apparatus of claim 1, further comprising:
a magnifying lens mounted within at least one of a tip portion of the tapered body and the second opening of the housing; and
an extractor arm actuation member movably attached to the housing, wherein movement of the extractor arm actuation member from an at-rest position toward a displaced position causes each one of said extractor arms to move from the respective retracted position toward the respective extended position;
wherein each one of said extractor arms is biased to the respective retracted position;
wherein said extractor arms are biased against an exterior face of the tapered body such that movement of said extractor arms toward the extended position causes each one of said extractor arms to follow a profile of the tapered body whereby tip portions of said extractor arms move closer together as each one of said extractor arms is moved from the respective retracted position toward the respective extended position; and
wherein said tip portions of each one of said extractor arms remain within a field of view through the viewing channel of the tapered body when each one of said extractor arms is moved between the respective retracted position and the respective extended position.

9. An earwax extracting otoscope, comprising:
a head assembly including a head assembly housing, a magnifying lens attached to the head assembly housing, spaced apart extractor arms movably attached to the head assembly housing and a tapered body attached to the head assembly housing, wherein a generally straight line of sight extends through the magnifying lens and a viewing passing of the tapered body, wherein each one of said extractor arms is movable between a retracted position and an extended position with respect to a tip portion of the tapered body and wherein the tapered body extends longitudinally between said spaced apart extractor arms;
a handle assembly including a handle assembly housing attached to the head assembly housing and a light source mounted within the handle assembly housing; and
an optical fiber attached between the light source and the head assembly housing for enabling a beam of light to be directed from the optical fiber through the viewing passage of the tapered body.

10. The earwax extracting otoscope of claim 9 wherein:
each one of said extractor arms is biased to the respective retracted position; and
said extractor arms are biased against an exterior face of the tapered body such that movement of said extractor arms toward the extended position causes each one of said extractor arms to follow a profile of the tapered body whereby tip portions of said extractor arms move closer together as each one of said extractor arms is moved from the respective retracted position toward the respective extended position.

11. The earwax extracting otoscope of claim 10 wherein said tip portions of said extractor arms remain within a field of view through the viewing channel of the tapered body when each one of said extractor arms is moved between the respective retracted position and the respective extended position.

12. The earwax extracting apparatus of claim 9 wherein tip portions of said extractor arms remain within a field of view through the viewing channel of the tapered body when each one of said extractor arms is moved between the respective retracted position and the respective extended position.

13. The earwax extracting otoscope of claim 9 wherein the magnifying lens is mounted within a tip portion of the tapered body.

14. The earwax extracting otoscope of claim 9 wherein:
the magnifying lens is mounted within an opening of the head assembly housing; and
the line of sight extends through the opening.

15. The earwax extracting otoscope of claim 9 wherein:
the head assembly includes an extractor arm actuation member movably attached to the housing; and
movement of the extractor arm actuation member from an at-rest position toward a displaced position causes each one of said extractor arms to move from the respective retracted position toward the respective extended position.

16. The earwax extracting otoscope of claim 9 wherein:
each one of said extractor arms is biased to the respective retracted position;
each one of said extractor arms is biased against an exterior face of the tapered body such that movement of each one of said extractor arms toward the respective extended position causes each one of said extractor arms to follow a profile of the tapered body whereby tip portions of each one of said extractor arms move closer together as each one of said extractor arms is moved from the respective retracted position toward the respective extended position;
the magnifying lens is mounted within one of a tip portion of the tapered body and an opening of the head assembly housing; the head assembly includes an extractor arm actuation member movably attached to the housing;
movement of the extractor arm actuation member from an at-rest position toward a displaced position causes each one of said extractor arms to move from the respective retracted position toward the respective extended position; and
said tip portions of said extractor arms remain within a field of view through the viewing channel of the tapered body when each one of said extractor arms is moved between the respective retracted position and the respective extended position.

17. A method for extracting earwax from within an ear canal, comprising:
inserting an invasive portion of an earwax extracting apparatus into an ear canal of a patient, wherein the invasive portion includes a respective tip portion of spaced apart extractor arms and a tip portion of a tapered body disposed between said extractor arms;
directing a beam of light through a viewing passage of the tapered body into the ear canal for enabling earwax therein to be viewed through the viewing passage of the tapered body;
causing said extractor arms to displace with respect to the tip portion of the tapered body for bringing said extractor arms into engagement with earwax in the ear canal; and
extracting the invasive portion of the earwax extracting apparatus from within the ear canal.

18. The method of claim 17 wherein causing said extractor arms to displace includes moving an extractor arm actuation member from an at-rest position toward a displaced position thereby causing each one of said extractor arms to move from the respective retracted position toward the respective extended position.

19. The method of claim 17 wherein:
causing said extractor arms to displace includes moving said extractor arms longitudinally with respect to a tip portion of each one of said extractor arms.

20. The method of claim 19 wherein moving said extractor arms includes maintaining tip portions of said extractor arms within a field of view through the viewing channel of the tapered body when each one of said extractor arms is moved between the respective retracted position and the respective extended position.
